(19) United States
(12) Patent Application Publication
(10) Pub. No.: US 2026/0305980 A1
(43) Pub. Date: Oct. 8, 2026

(54) TECHNOLOGY CONFIGURED TO ENABLE DETERMINATION OF SLEEP APNOEA RISK FACTORS FOR SUBJECT BASED ON ARTEFACTS OF TIME-SERIES EYELID AMPLITUDE DATA COLLECTED DURING CONSCIOUS STATE

(71) Applicant: SDIP Holdings PTY LTD, Cremorne, Victory (AU)

(72) Inventors: Scott Coles, Cremorne, Victoria (AU); Trefor Morgan, Cremorne, Victoria (AU); Hedi Ziv, Cremorne, Victoria (AU); Ali Almasi, Cremorne, Victoria (AU); Vipul Jain, South Morang, Victoria (AU)

(21) Appl. No.: 19/476,480

(22) PCT Filed: Apr. 17, 2024
(86) PCT No.: PCT/AU2024/050369

§ 371 Date: Oct. 17, 2025

(30) Foreign Application Priority Data
Apr. 18, 2023 (AU) 2023901148

(51) Int. Cl. A61B 5/11; A61B 5/00; G06V 20/59; G06V 40/18

(52) U.S. Cl. CPC A61B 5/1103; A61B 5/1128; A61B 5/4818; A61B 5/6893; A61B 5/7282; G06V 20/597; G06V 40/193

(57) ABSTRACT

Technology is configured to enable determination of sleep apnea risk factors for a subject based on artifacts of time-series eyelid amplitude data collected during conscious state (i.e., during wakefulness). Risk factors may include a metric representative of a risk that the subject suffers from sleep apnea (e.g., OSA), and in some cases metrics representative of a predicted level of severity associated with that sleep apnea. Embodiments are described by reference to testing technology and methods, which are conducted while a subject is conscious and performing a known task, with eyelid amplitude data collected during testing being processed to extract a plurality of first derivative artifacts that are used for the purposes of sleep apnea risk determinations.

### CROSS-REFERENCE TO RELATED APPLICATIONS

This application is a national phase entry under 35 U.S.C. § 371 of International Patent Application PCT/AU2024/050369, filed Apr. 17, 2024, designating the United States of America and published as International Patent Publication WO 2024/216336 A1 on Oct. 24, 2024, which claims the benefit under Article 8 of the Patent Cooperation Treaty of Australian Patent Application Serial No. 2023901148, filed Apr. 18, 2023.

### TECHNICAL FIELD

The present disclosure relates, in various embodiments, to technology configured to enable determination of sleep apnea risk factors for subject based on artifacts of time-series eyelid amplitude data collected during conscious state (i.e., during wakefulness). Risk factors may include a metric representative of a risk that the subject suffers from sleep apnea (e.g., OSA), and in some cases metrics representative of a predicted level of severity associated with that sleep apnea. Embodiments are described by reference to testing technology and methods that are conducted while a subject is conscious and performing a known task, with eyelid amplitude data collected during testing being processed to extract a plurality of first derivative artifacts that are used for the purposes of sleep apnea risk determinations. However, it will be appreciated that the technology is not limited as such, and has application in a broader range of context.

### BACKGROUND

Any discussion of the background art throughout the present disclosure should in no way be considered as an admission that such art is widely known or forms part of common general knowledge in the field.

The most common form of sleep apnea is OSA (Obstructive Sleep Apnea). If left untreated can cause stroke, heart disease, diabetes, high blood pressure etc. OSA is believed to affect approximately 20% of US adults, of whom about studies indicate that around 80% to 90% are undiagnosed.

The current gold standard for OSA diagnosis is Polysomnography (PSG), measured during sleep. Other testing methods are beginning to be increasingly accepted in the medical sleep space, including home-based sleep testing. These approaches, while useful, have historically been unsuccessful in substantively reducing the extent of undiagnosed cases. In particular, there remain substantial practical and educational barriers to many people being tested.

### BRIEF SUMMARY

It is an object of the present disclosure to overcome or ameliorate at least one of the disadvantages of the prior art, or to provide a useful alternative.

Example embodiments are described below in the sections entitled “detailed description” and “claims.”

Reference throughout this disclosure to “one embodiment,” “some embodiments,” or “an embodiment” means that a particular feature, structure or characteristic described in connection with the embodiment is included in at least one embodiment of the present disclosure. Thus, appearances of the phrases “in one embodiment,” “in some embodiments,” or “in an embodiment” in various places throughout this disclosure are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms “comprising,” “comprised of,” or “which comprises” is an open term that means including at least the elements/features that follow, but not excluding others. Thus, the term “comprising,” when used in the claims, should not be interpreted as being limitative to the means or elements or steps listed thereafter. For example, the scope of the expression a device comprising A and B should not be limited to devices consisting only of elements A and B. Any one of the terms “including,” “which includes,” or “that includes” as used herein is also an open term that also means including at least the elements/features that follow the term, but not excluding others. Thus, “including” is synonymous with and means “comprising.”

As used herein, the term “exemplary” is used in the sense of providing examples, as opposed to indicating quality. That is, an “exemplary embodiment” is an embodiment provided as an example, as opposed to necessarily being an embodiment of exemplary quality.

### BRIEF DESCRIPTION OF THE DRAWINGS

Embodiments of the present disclosure will now be described, by way of example only, with reference to the accompanying drawings in which:

FIG. 1A illustrates an in-vehicle monitoring system according to one embodiment.

FIG. 1B illustrates an in-vehicle monitoring system according to one embodiment.

FIG. 2 illustrates a method according to one embodiment.

### DETAILED DESCRIPTION

The present disclosure relates, in various embodiments, to technology configured to enable determination of sleep apnea risk factors for subject based on artifacts of time-series eyelid amplitude data collected during conscious state (i.e., during wakefulness). Risk factors may include a metric representative of a risk that the subject suffers from sleep apnea (e.g., OSA), and in some cases metrics representative of a predicted level of severity associated with that sleep apnea. Embodiments are described by reference to testing technology and methods that are conducted while a subject is conscious and performing a known task, with eyelid amplitude data collected during testing being processed to extract a plurality of first derivative artifacts that are used for the purposes of sleep apnea risk determinations. However, it will be appreciated that the technology is not limited as such, and has application in a broader range of context.

In overview, technology described below passive monitoring of a human subject, (for example, passive monitoring while they perform “ordinary” day-to-day tasks, and/or active monitoring whereby they perform known/designated tasks), thereby to generate a metric representative of a sleep apnea risk factors. For example, this metric may be representative of a risk of obstructive sleep apnea (OSA), optionally in combination with a severity level (e.g., a risk of moderate or intense OSA). However, it will be recognized that the technology may in some embodiments be additionally and/or alternately applied in relation to other forms of sleep apnea. More generally, technology described herein is, in some embodiments, applied in respect of other forms of sleep hygiene, such as ESS, excessive daytime sleepiness, and the like.

For the purposes of embodiments described below, the metric representative of sleep apnea risk factors is described by reference an Obstructive Sleep Apnea Metric, or OSAM. This term is introduced for the purpose of convenience in description only, and that the OSAM is optionally replaced with any other metric representative of sleep apnea risk factors (OSA and/or otherwise).

In some embodiments, the metric representative of sleep apnea risk factors is additionally associated with a severity metric. For example, the following possible example OSAM output labels are provided as guidance for the reader: “OSA likelihood detected”; “OSA likelihood not detected”; “mild OSA likelihood detected”; “moderate OSA likelihood detected”; “severe OSA likelihood detected”; “high OSA likelihood detected”; “medium OSA likelihood detected”; “low OSA likelihood detected”; and/or combinations of the foregoing. It will be appreciated that these are examples only, and that such labels are associated numerical values provided via a data processing system as described further below.

The concepts of “sleep apnea risk factors,” “sleep apnea risk,” “sleep apnea risk metrics,” and the like are used in the context of objective data that provides an indication as to a likelihood that a person suffers from sleep apnea (such as OSA). This may be in practice presented, for instance, as a determination that a person is an OSA sufferer (e.g., that a person suffers from mild OSA, moderate OSA or severe OSA). However, it will be appreciated that predictions as to a person's sleep apnea status made by technology described herein are not definitive, and rather they are probabilistic (hence the use of the term “risk”). In another example, an output might be presented as, for instance, “your results conclude that there's a >80% chance/probability that you suffer from moderate OSA.”

The monitoring includes collection of time-series eyelid amplitude data, which is subsequently processed thereby to detect individual blink events (including eyelid movement events, which occur in the context of blinking) and, for each of the individual blink events, generate a plurality of blink artifacts. The blink artifacts include blink artifacts derived from of time-series amplitude data. It has been identified that a range of such artifacts are of particular use for the purposes of identifying sleep apnea risks (e.g., OSA) for a particular individual. The blink artifacts derived from time-series amplitude data preferably include one or more of the following:
A combination of artifacts that n^{th} and n+1^{th} derivatives of the time-series amplitude data, where n is an integer greater than or equal to zero. This is referred to as use of “multi-ordered derivative artifacts” for the purposes of this disclosure. It will be appreciated that oth derivative artifacts may include the likes of maximum amplitude, relative amplitude, relative position, and the like.
A combination of artifacts including at least one artifact derived from a first derivative of the time-series amplitude data. For example, artifacts derived from a first derivative of the time-series amplitude data may include eyelid closure velocities and/or eyelid opening velocities, positive and negative amplitude-to-velocity ratios, and many others. Those familiar with blepharometric analysis techniques will appreciate a wide range of such artifacts.
A combination of artifacts including a plurality of temporal artifacts. The term “temporal artifact” is used to describe a data artifact for a blink event that is defined by a time period between two objectively identifiable sub-events within the blink event. For example, temporal artifacts used in the context of blepharometric analysis include one or more of: blink total duration; inter-eyelid movement event duration (positive and/or negative); zero crossing intervals (positive and/or negative); duration of ocular quiescence (positive and/or negative); blink eyelid closed duration. Those familiar with blepharometric analysis techniques will appreciate a wide range of such artifacts.
A combination of one or more temporal artifacts and one or more multi-ordered derivative artifacts.
A combination of any of the above with one or more inter-blink artifacts. The term “inter-blink artifact” is used to describe artefacts that are determined based on a combination of two or more detected blink events in the time-series amplitude data, for example, using objectively defined start/end points for each blink event. Such “inter-blink artifacts” include blink rate/frequency, inter-blink duration, and the like. For the purposes of this disclosure, the term “temporal artifact” specifically excludes any “inter-blink artifacts” from a definitional perspective. This clearly differentiates temporal artifacts from individual blink events from blink frequency metrics and the like.

Additional data artifacts may optionally, in some embodiments, be used in combination with those derived from the time-series amplitude data. For example, in some embodiments, data artifacts are extracted from monitoring of eye movement.

Example blink artifacts from the various artifact categories (multi-ordered derivative, temporal and inter-blink) are provided in the section further below headed “example blink artifacts.”

In a preferred embodiment, the monitoring is facilitated by existing in-vehicle driver monitoring systems. Some examples of such systems are presently configured to enable generation of time-series blink amplitude data, for example, based on image processing of frames generated by a video capture system (these may include facial image data, for instance, image data that includes at least part of a face). Such technology has previously been deployed for the purposes of detecting drowsiness in drivers. The technology described herein allows for such existing systems to be applied thereby to enable determination of sleep apnea risks for vehicle operators. This, in some cases, is achieved via installation of additional software in an existing driver monitoring system. However, data processing required for the purposes of determination of sleep apnea risks is optionally performed by or in conjunction with processing components external of the vehicle.

While the present disclosure focuses, in particular, on application of the relevant processing technology in the context of in-vehicle driver monitoring systems, alternate hardware environments may be used for eyelid amplitude data collection. For example, this may include other in-vehicle monitoring systems such as occupant monitoring systems (e.g., in cars, airplanes, trains and other mass transport vehicles). Outside of a vehicle environment, this may include the use of cameras that monitor the face of a subject using a computer, mobile device, or other hardware including a screen, thereby to collect the eyelid amplitude data while the subject uses that device (including either or both of unstructured usage and usage comprising performance of designated tasks; in some embodiments, eyelid movement data is collected during videoconferencing as an additional functionality for a videoconferencing platform). Data collection hardware may include cameras and/or wearable devices (for example, infrared oculography hardware, and VR/AR goggles and/or headsets). In some embodiments, contextual data is collected, thereby to provide context to eyelid movement data. For example, in videoconferencing, contextual data may include whether a subject is speaking or listening. In the context of driving, this may include whether the vehicle is in motion (and optionally other factors such as driving speed, urban/freeway style of driving, and so on).

The time-series data input representative of eyelid amplitude is collected from the subject during a “testing period.” This testing period is preferably an “unstimulated” period, meaning that there are no controlled stimuli delivered to the user as part of the testing protocol to trigger blinks, or otherwise instruct voluntary blinks (i.e., monitoring is performed without deliberate stimulation of blink events). That is, the monitoring is employed such that blink events detected for data corresponding to the testing are likely to be spontaneous involuntary blinks. It will be appreciated by those skilled in the art that characteristics of spontaneous involuntary blinks are capable of revealing a range of neurological conditions, given physiological mechanisms that control the relevant movements.

In some embodiments, OSAM data for a given subject is tracked over an extended period of time, thereby to monitor changes in metric calculations. This is optionally applied thereby to assess the effectiveness of sleep apnea treatments to which the subject is subjected. These treatments may include medical and/or non-medical interventions. In some embodiments, the OSAM includes a value defined on a scale representative of predicted severity of OSA, allowing a user to track their progress over time in terms of susceptibility/severity.

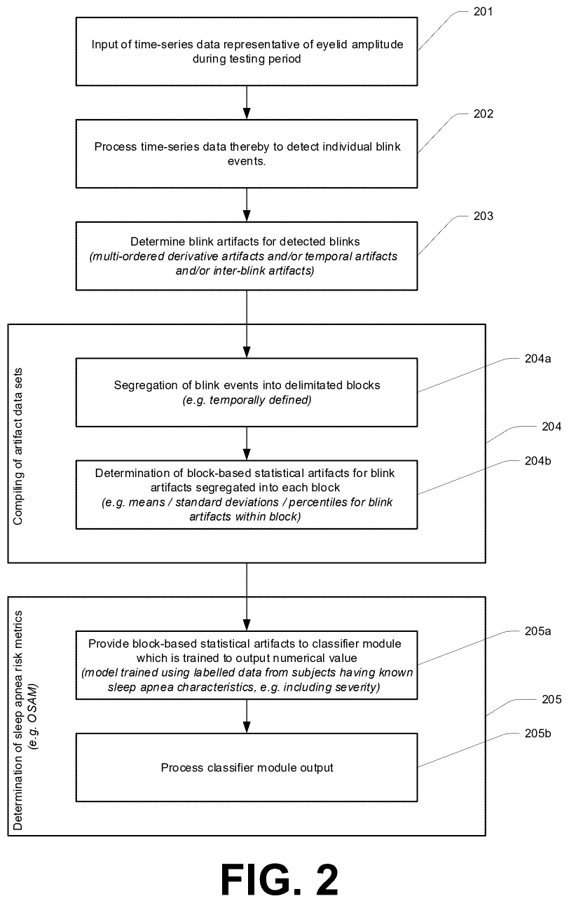

Referring to FIG. 2, one embodiment provides a method for determining a metric representative of sleep apnea risk factors for a human subject.

Block 201 represents a process including receiving time-series data input representative of eyelid amplitude for the subject during a testing period. For example, this may be derived from image processing techniques such as those disclosed in PCT patent publication WO2020082125, whereby image frames collected by a capture device are processed thereby to define eyelid amplitude data for each frame and from that generate time-series data (optimally including processes such as interpolation thereby to generate a sampling rate greater than a camera frame rate). Alternately, hardware such as that used for infrared oculography may be employed.

Block 202 represents a process including processing the time-series data thereby to detect a plurality of blink events. A process for identifying blink events may include applying algorithms to time-series data thereby to identify specific markers that objectively delineate the start and end of a blink, for example, based on changes and/or zero values for one or more multi-ordered derivative artifacts. In a simple case, this is used to identify a point where eyelid closure commences and a point where eyelid re-opening completes, and define a blink event as the time-series data between those points. Definitions for what constitutes start and end points for blink events may vary between embodiments. Blink events may be designated with sequential identifiers, thereby to facilitate analysis that is based on processing of data relating to consecutive blink events (e.g., inter-event artifacts).

Block 203 represents, for each blink event, determining a plurality of blink event artifacts, for example, multi-ordered derivative artifacts and/or temporal artifacts (optionally in combination with inter-blink artifacts).

Block 204 represents a process including compiling one more artifact data sets, wherein each artifact data set includes blink artifacts (including blink artifacts derived from a first derivative of time-series amplitude). Each artifact data set corresponds to a defined plurality of blink events during the testing period. In the illustrated example, block 204 includes two distinct sub-processes:
Block 204a: segregating blink events into delineated blocks. These may be temporally delineated blocks. For example, each delineated block corresponds to a X-second time period, wherein X is a value between 30 and 240 (or more preferably between 45 and 120). A preferred embodiment makes use of 60-second blocks. Alternately, segregation may be based on other factors, for example, segregation such that each block contains an equal (e.g., predefined) number of blink events, for example, between about 10 and 30 blink events (although other block sizes may be used).
Block 204b: for each delineated block, performing statistical analysis of the blink artifacts thereby to derive block-specific blink artifact statistics. These are referred to as “block-based statistical artifacts,” being a second order collection of artifacts derived from multi-ordered derivative, temporal and/or inter-blink artifacts for blink events delimitated into a given block.

The process of deriving block-specific blink artifact statistics for each delineated block preferably includes applying a set of statistical analysis algorithms to the blink artifacts derived at block 203 for blink events corresponding to that block. In a preferred embodiment this includes Mean, Standard Deviation, Percentiles, and further Higher-Order Statistics.

Block 205 represents a process including processing each of the artifact data sets thereby to determine a metric representative to a risk of sleep apnea. This preferably includes the following sub-processes:
Block 205a: providing each artifact data set to a classifier module thereby to derive an output value. This output value is, in some embodiments, a value between zero and one.
Block 205b: Determining the metric based on processing of a plurality of output values derived from the plurality of artifact data sets. For example, where values are outputted by the process of block 205a, the metric may be based on output of the first layer. This may result in a risk metric defined on a scale (for example, representative of low risk to high risk), defined in a binary manner (for example, “not determined to be at risk” or “determined to be at risk”), and/or include information relating to predicted severity level (e.g., mild/moderate/severe sleep apnea). Majority decisions, heuristics and/or other techniques may be used to determine the metric at this stage.

The classifier module preferably utilizes a machine learning model (or other form of technology, such as AI technology, which enables classification). Various machine learning models may optionally be trained using various features engineered and described above. An instance of such models includes a layered architecture of a Random Forest (RF) model stacked on top of multiple Decision Tree (DT) models. Other model instances can include, but are not limited to, more black box models such as deep neural network (DNN) models like Deep Convolutional Neural Networks (DCNN), Recurrent Neural Networks (RNN) and Long-Short-Term Memory (LSTM) networks. Those skilled in the art will understand how such machine learning models may be trained based on features and training data sets. The training data sets may be labelled based on a binary “sleep apnea sufferer” or “no sleep apnea” labelling protocol, or via a more complex protocol that separates “sleep apnea sufferer” into condition levels such as mild, moderate and severe.

The manner by which the final risk metric is communicated to the subject varies between embodiments based upon the overarching technological framework. For example, decision tree and/or other algorithmic logic may be configured to convert a numerical classifier output into one or more descriptive labels belonging to a predefined schema.

### Example Hardware Frameworks

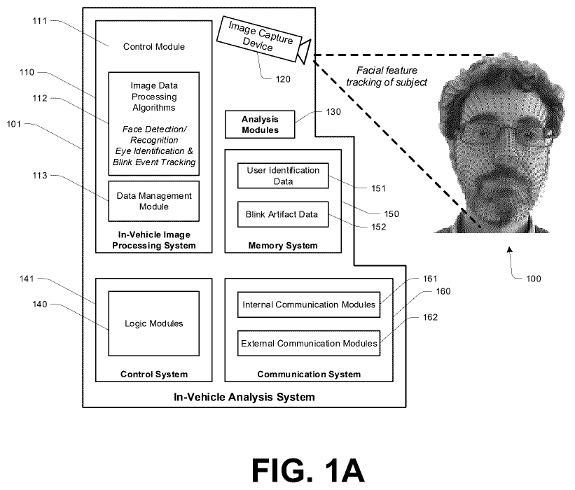

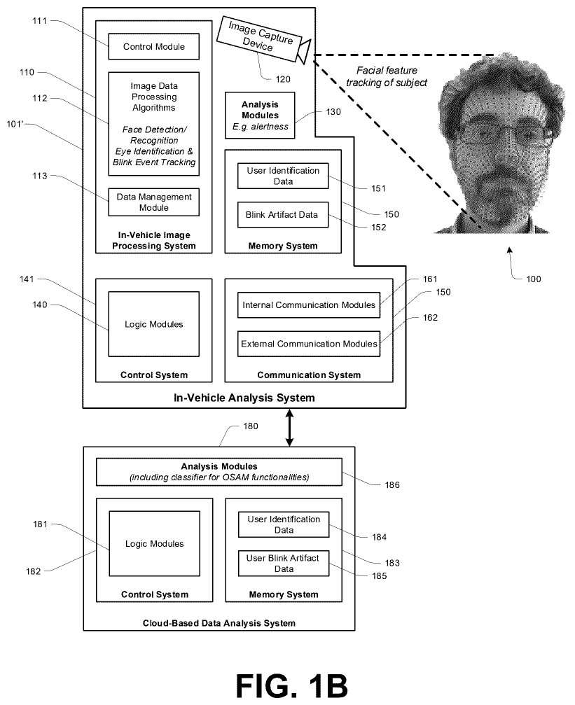

FIG. 1A illustrates an example in-vehicle monitoring system configured to enable prediction of sleep apnea risk factors as described herein. It will be appreciated that this is an example only, and that a wide range of known commercial systems are configured to perform eyelid position tracking may be modified for the purposes of assessing sleep apnea risk factors as discussed herein. For example, such known systems already record time-series data for eyelid amplitude, and in some cases extract a range of artifacts from such data. Known systems such as these are optionally provided with additional software functionality (locally and/or in cloud-hosted systems) thereby to facilitate processing leading to generation of sleep apnea relevant metric such as an OSAM based on data already being collected from a driver.

The system of FIG. 1A includes an image capture device 120. This may include substantially any form of appropriately sized digital camera, preferably a digital camera with a frame rate of over 30 frames per second. Higher frame rate cameras are advantageous, given that with enhanced frame rate comes an ability to obtain higher resolution data for eyelid movement. For example, other embodiments use cameras with frame rates of between 60 and 240 frames per second.

Device 120 is positioned to capture a facial region of a subject 100. Device 120 is in one embodiment installed in a region of a vehicle in the form of an automobile, for example, on or adjacent the dashboard, windscreen, or visor, such that it is configured to capture a facial region of a driver. In another embodiment, device 120 is positioned on or adjacent the dashboard, windscreen, or visor, such that it is configured to capture a facial region of a front seat passenger. In another embodiment, device 120 is positioned in a region such as the rear of a seat such that it is configured to capture a facial region of a back-seat passenger. In some embodiments, a combination of these is used, thereby to enable eyelid movement monitoring for both a driver and one or more passengers.

Although the system of FIG. 1A (and other systems) are described by reference to a vehicle in the form of an automobile, it will be appreciated that a system as described is also optionally implanted in other forms of vehicles, including mass-transport vehicles such as passenger airplanes, buses/coaches, and trains. In such embodiments, there are preferably one or more analysis systems each supporting a plurality of image capture devices, each positioned to capture a respective passenger. Furthermore, the technology disclosed herein may also be implemented away from vehicles, for example, to allow for OSA assessments to be performed in pharmacies, laboratories and at home (for example, using computing devices such as PCs, laptops, smartphones and tablets with a screen that provides a “task” for a subject, and a camera to record eyelid movement while the task is performed).

An in-vehicle image processing system 110 is configured to receive image data from image capture device 120 (or multiple devices 120), and process that data thereby to generate time-series eyelid amplitude data. A control module 111 is configured to control device 120, operation of image data processing, and management of generated data. This includes controlling operation of image data processing algorithms, which are configured to:
(i) Identify that a human face is detected.
(ii) In embodiments where subject identification is achieved via facial recognition algorithms (which is not present in some embodiments, for example, embodiments that identify a subject via alternate means), perform a facial recognition process thereby to identify the subject. This may include identifying a known subject based on an existing subject record defined in user identification data 151 stored in a memory system 150, or identifying an unknown subject and creating a new subject user identification data 151 stored in a memory system 150.
(iii) In a detected human face, identify an eye region. In some embodiments, the algorithms are configured to track one eye region only; in other embodiments, both eye regions are tracked thereby to improve data collection.
(iv) Identify, in the eye region(s), presence and movement of an eyelid. For example, in a preferred embodiment this is achieved by way of recording an eyelid position relative to a defined “closed” position against time. This allows generation of time-series eyelid amplitude data in the form of eyelid position (amplitude) over time. It will be appreciated that such data provides for identification of events (for example, blink events) and velocity (for example, as a first derivative of position against time).

Algorithms 112 optionally operate to extract artifacts from the time-series eyelid amplitude data, for example, temporal artifacts, multi-ordered derivative artifacts and in some cases additionally inter-blink artifacts. It will be appreciated, however, that extraction of such artifacts may occur in downstream processing.

A data management module 113 is configured to coordinate storage of data generated by algorithms 112 in user time-series eyelid amplitude data 152. In some embodiments, the function of module 113 includes determining whether a set of generated time-series eyelid amplitude data meets threshold data quality requirements for storage, for example, based on factors including a threshold unbroken time period for which eyelid tracking is achieved and time-series eyelid amplitude data is generated.

As an optional feature, memory system 150 includes user identification data 151 for one or more users. As noted, in some embodiments, system 101 is configured to collect and analyze time-series eyelid amplitude data for only a single user (for instance, the primary driver of a vehicle) and includes identification data to enable identification of only that user. In other embodiments, system 101 includes functionality to collect and analyze time-series eyelid amplitude data for multiple users, and includes identification data to enable identification of any of those users (and optionally, as noted above, defining of a new record for a previously unknown user). The identification data may include login credentials (for example, a user ID and/or password) that are inputted via an input device. Alternately, the identification data may be biometric, for example, using facial recognition as discussed above or an alternate biometric input (such as a fingerprint scanner). In some embodiments, this leverages an existing biometric identification system of a device/vehicle/other.

Blink artifact data 152 includes is configured to time-series eyelid amplitude data for one or more testing periods, and optionally/additionally store associated blink artifact data and/or delineated block-specific blink artifact data sets as described herein (although noting that various processing steps may instead be performed externally of system 101).

Analysis modules 130 are configured to cause analysis of one or more artifact data sets, each artifact data set being derived from the blink artifacts, including blink artifacts derived from a first derivative of time-series amplitude, for a defined plurality of blink events during a given testing period. This may include analysis relating to driver alertness monitoring, and associated notifications and/or intervention management. In this embodiment, module 130 additionally causes processing each of a plurality of the artifact data sets (e.g., via a classifier, which may be cloud hosted) thereby to determine a metric representative to a risk of sleep apnea, such as an OSAM.

System 101 additionally includes a communication system 160, which is configured to communicate information from system 101 to human users. This may include internal communication modules 161 that provide output data via components installed in the vehicle, for example, an in-car display, warning lights, and so on. External communication modules 162 are also optionally present, for example, to enable communication of data from system 101 to user devices (for example, via BLUETOOTH®, Wi-Fi, or other network interfaces), optionally by email or other messaging protocols. In this regard, communication system 160 is optionally configured to facilitate communication results of analysis by analysis modules 130.

A control system 140 included logic modules 141 that control overall operation of system 140. This includes execution of logical rules thereby to determine communications to be provided in response to outputs from analysis modules 130. For example, this may include in-vehicle notifications. These in-vehicle notifications might be unrelated to sleep apnea assessment, for example, where blink event data is processed for more time-critical purposes such as assessing impairment/drowsiness. An OSAM or other apnea-related metric may be communicated via the vehicle, or that function may be responsibility of a separate cloud-based system. It will be appreciated that these are examples only, and logic modules 141 are able to provide a wide range of functionalities thereby to cause system 101 to act in a predefined manner.

FIG. 1B illustrates a further embodiment in the form of system 101′, which includes various common features with the embodiment illustrated in FIG. 1A. In general terms, in some embodiments, external communication modules 162 facilitate communication with a remote server device in the form of a data analysis system 180, which performs analysis of time-series eyelid amplitude data analysis. This communication may rely on various intermediary devices to facilitate network connectivity (e.g., including a smartphone).

System 180 includes a control system 182 and logic modules 181, which are provided by computer executable code executing across one or more computing devices thereby to control and deliver functionalities of system 180.

System 180 additionally includes a memory system 183, which stores user testing data 185 including and/or derived from time-series eyelid amplitude data collected via system 101′, enabling such data 185 to be passed to classifier modules for the purposes of generating an OSAM for each testing period. In some cases, memory system 183 optionally includes user identification data 184 thereby to facilitate tracking of changes in user-specific OSA metrics over time, for example, to assess effectiveness of medical and/or non-medical treatment regimens.

System 180 additionally includes analysis modules 186. In this example, analysis modules 186 perform analysis via classifier modules, including a classifier module that is configured to facilitate generation of an OSAM as described herein. The OSAM is optionally communicated to the relevant subject via a range of technologies, including email and other electronic messaging platforms. Analysis modules 130 perform in-vehicle algorithmic analysis, for example, in relation to driver impairment/alertness/drowsiness. In this manner, data collected for the purposes of in-vehicle analysis as part of a driver monitoring system is complemented by cloud-based processing that delivers metrics relevant to sleep apnea.

### Example Blink Artifacts

Provided below are examples of artifacts belonging to various artifact categories that are optionally used in any of the preceding embodiments.

Example multi-ordered derivative artifacts include:
Relative amplitude.
Relative position.
Maximum amplitude.
Minimum amplitude.
Blink start position.
Blink end position.
Maximum velocities (opening and/or closing).
Zero crossing index (positive and/or negative).
Amplitude-to-velocity ratios (positive and/or negative).
Composite artifacts generated from a linear or nonlinear combination of two or more the above and/or other artifacts.

Example temporal artifacts include:
Blink total duration (BTD), which is preferably measured as a time between commencement of closure movement that exceeds a defined threshold and completion of subsequent opening movement.
Inter-event durations (positive and/or negative), which may be used to measure temporal artifacts for a period during which the eyelid is moving in a closing direction and/or opening direction.
Blink eyelid closed duration (BECD).

Example inter-blink artifacts include:
Blink frequency.
Blink rate.
Inter-blink event duration.
Blink free interval (BFI).
Saccade rate.
Cumulative Time No Movement.

Example block-based statistical artifacts include:
Mean of any one or more blink artifact values.
Standard deviation for any one or more blink artifact values.
Percentile measures for any one or more blink artifact values.
Third-order moment statistic, i.e., skewness.
Fourth-order moment statistic, i.e., kurtosis.

These are examples only, and others may be used.

### Conclusions and Interpretation

It will be appreciated that the above disclosure provides technology configured to enable determination of sleep apnea risk factors for subject based on artifacts of time-series eyelid amplitude data collected during conscious state. This offers distinct advantages over existing technologies, in the sense that testing is far less obstructive/inconvenient, being performed during a wakeful state, optionally while using a computing device, watching a screen, and/or operating a vehicle. Furthermore, the technology allows for convenient monitoring of sleep apnea risk factors over time, thereby to enable assessment of effectiveness of treatments that are applied.

It should be appreciated that in the above description of exemplary embodiments of the present disclosure, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this disclosure.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the present disclosure may be practiced without these specific details. In other instances, well-known methods, structures and techniques have not been shown in detail in order not to obscure an understanding of this disclosure.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B that may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the present disclosure, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present disclosure.

1. A method determining a metric representative of sleep apnea risk factors for a human subject, the method including:
receiving time-series data input representative of eyelid amplitude for the subject during a testing period;
processing the time-series data thereby to detect a plurality of blink events;
for each blink event, determining a plurality of blink event artifacts including blink artifacts derived from time-series amplitude data;
compiling one more artifact data sets, wherein each artifact data set is derived from the blink artifacts, including blink artifacts derived from the time-series amplitude data for a defined plurality of blink events during the testing period; and
processing each of the artifact data sets thereby to determine a metric representative to a risk of sleep apnea.

2. The method of claim 1, wherein the time-series data input representative of eyelid amplitude for the subject during a testing period is collected via a hardware arrangement that is configured to conduct data capture while the subject is engaging in a known task.

3. The method of claim 2, wherein the known task is operation of a motorized vehicle.

4. The method of claim 3, wherein the time-series data input representative of eyelid amplitude for the subject during a testing period is collected via a driver or occupant monitoring system.

5. The method of claim 4, wherein the driver monitoring system includes a camera configured to capture image frames of a facial region of the subject, and wherein the facial image frames are processed thereby to derive the time-series data input representative of eyelid amplitude.

6. The method of claim 2, wherein the known task is utilization of a computer or mobile device.

7. The method of claim 1, wherein the time-series data input representative of eyelid amplitude is collected using a camera system that is configured to collect facial image frames, wherein facial image frames collected via the camera system are processed thereby to derive the time-series data input representative of eyelid amplitude.

8. The method of claim 1, wherein the time-series data input representative of eyelid amplitude is collected using a wearable device.

9. (canceled)

10. The method of claim 1, wherein the step of compiling one more artifact data sets includes: (i) defining a group of blink artifacts for each of the detected blink events; (ii) segregating detected blink events into delineated blocks; and (iii) for each delineated block, performing statistical analysis of the blink artifacts thereby to derive block-specific blink artifact statistics.

11. The method of claim 10, wherein each artifact data set includes the block-specific blink artifact statistics for a respective one of the delineated blocks.

12. The method of claim 10, wherein the delineated blocks are defined based on time periods.

13. (canceled)

14. (canceled)

15. The method of claim 10, wherein the delineated blocks are defined based on a prescribed number of blink events, such that each delineated block corresponds to an equal number of blink events.

16. The method of claim 1, wherein processing each of the artifact data sets thereby to determine a metric representative to a risk of sleep apnea includes, where there is a plurality of artifact data sets: (i) providing each artifact data set to a classifier module thereby to derive an output value; and (ii) determining the metric based on processing of a plurality of output values derived from the plurality of artifact data sets.

17. (canceled)

18. The method of claim 1, wherein the metric representative of sleep apnea risk factors for the human subject is a metric representative of obstructive sleep apnea risk factors.

19. The method of claim 1, wherein the blink artifacts include multi-ordered derivative artifacts.

20. The method of claim 1, wherein the blink artifacts include at least one multi-ordered derivative artifact and at least one temporal artifact.

21. The method of claim 1, wherein the blink artifacts include a combination of two or more of the following:
at least one multi-ordered derivative artifact, at least one temporal artifact; and at least one inter-blink artifact.

22. The method of claim 1, wherein the blink artifacts include one or more first-ordered derivative artifacts.

23. (canceled)

24. (canceled)

25. A method for determining a sleep apnea risk factors metric via passive monitoring of a conscious subject without deliberate stimulation of blink events.

26.-28. (canceled)

29. A method determining a metric representative of sleep hygiene for a human subject, the method including:
receiving time-series data input representative of eyelid amplitude for the subject during a testing period;
processing the time-series data thereby to detect a plurality of blink events;
for each blink event, determining a plurality of blink event artifacts including blink artifacts derived from time-series amplitude data;
compiling one more artifact data sets, wherein each artifact data set is derived from the blink artifacts, including blink artifacts derived from the time-series amplitude data for a defined plurality of blink events during the testing period; and
processing each of the artifact data sets thereby to determine a metric representative to sleep hygiene.

30. (canceled)
